(19) United States
(12) Patent Application Publication
(10) Pub. No.: US 2026/0207883 A1
(43) Pub. Date: Jul. 23, 2026

(54) CATHETER LUER ASSEMBLY

(71) Applicant: Affera, Inc., Newton, MA (US)

(72) Inventor: Marissa A. Metcalf, Galway (IE)

(21) Appl. No.: 19/454,659

(22) Filed: Jan. 21, 2026

Related U.S. Application Data
(60) Provisional application No. 63/748,701, filed on Jan. 23, 2025.

(51) Int. Cl. A61M 25/00; A61M 39/10; A61M 39/22

(52) U.S. Cl. CPC A61M 25/0009; A61M 25/0097; A61M 39/10; A61M 39/22; A61M 2207/00

(57) ABSTRACT

In one or more examples, this disclosure is directed to a luer assembly for a catheter assembly, the luer assembly comprising: an inner tube extending along a longitudinal axis; an outer tube disposed over the inner tube and extending along the longitudinal axis from a proximal end to a distal end; a sliding mechanism coupled to the outer tube; and a luer disposed around the proximal end of the outer tube, wherein the outer tube, the sliding mechanism, and the luer is overmolded over the inner tube.

The application claims the benefit of U.S. Provisional Patent Application No. 63/748,701, filed Jan. 23, 2025 and titled “CATHETER LUER ASSEMBLY”, the entire contents of which is incorporated herein by reference.

### TECHNICAL FIELD

The disclosure is related to catheters including handle assemblies for catheters.

### BACKGROUND

A catheter may be advanced over a guidewire to a target location within a patient. A handle at a proximal end of the catheter may be actuated by a clinician to control movement of the catheter within the patient and/or to position therapy delivery elements on the catheter within the patient. The handle may include a luer assembly which may receive the guidewire for insertion into the body of the patient.

### SUMMARY

The present disclosure is directed to devices, systems, and methods for forming a luer assembly via an overmolding technique. The luer assembly may include a luer, an outer tube, a sliding mechanism, and an inner tube. The luer assembly may be a part of a handle of a catheter (e.g., an over-the-wire catheter). The luer, the outer tube, and the sliding mechanism may be overmolded over the inner tube. The overmolded components may retain inner tube without requiring the use of adhesives to connect the components together, e.g., such as in other luer assemblies. The overmolded components (e.g., luer, outer tube, and sliding mechanism) may define a single monolithic element around at least a portion of the inner tube.

Forming the luer assembly via the overmolding technique may provide several advantages over other luer assemblies. The overmolded components may increase a contact surface area between the components and the inner tube, e.g., thereby providing improved sealing and/or tensile performance by the luer assembly compared to other luer assemblies. Use of the overmolding technique may simplify a manufacturing process for the luer assembly. The overmolded luer assemblies described herein may also provide an improved ability to inhibit unintended flow of fluids into or out of the inner tube of the luer assembly.

In some examples, this disclosure describes a luer assembly for a catheter assembly, the luer assembly comprising: an inner tube extending along a longitudinal axis; an outer tube disposed over the inner tube and extending along the longitudinal axis from a proximal end to a distal end; a sliding mechanism coupled to the outer tube; and a luer disposed around the proximal end of the outer tube, wherein the outer tube, the sliding mechanism, and the luer are overmolded over the inner tube.

In some examples, this disclosure describes a system comprising: a luer assembly comprising: an inner tube extending from an inner tube proximal end to an inner tube distal end along a longitudinal axis; a luer overmolded around the inner tube proximal end of the inner tube; an outer tube overmolded around the inner tube and extending distally from a luer distal end of the luer; and a sliding mechanism overmolded around the inner tube and coupled to the outer tube, wherein the luer, the outer tube, and the sliding mechanism define a monolithic component.

In some examples, this disclosure describes a method of manufacturing a luer assembly, the method comprising: placing an inner tube and a center pin within a mold, wherein the inner tube and the center pin are aligned along a longitudinal axis, and wherein a distal end of the center pin is inserted into a proximal end of the inner tube; inserting a polymer resin into the mold and round the inner tube and the center pin to overmold the polymer resin around the inner tube and the core pin to form: a luer around an inner tube proximal end of the inner tube, an outer tube around the inner tube and coupled to a luer distal end of the luer, and a sliding mechanism around the inner tube and coupled to the outer tube; and removing the center pin from within the luer assembly.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example luer assembly.

FIG. 2 is a conceptual diagram illustrating an example partially cross-section of the luer assembly of FIG. 1.

FIG. 3 is a conceptual diagram illustrating an example cross-section of the luer of the luer assembly of FIG. 1.

FIG. 4 is a conceptual diagram illustrating an example sliding mechanism of the luer assembly of FIG. 1.

FIG. 5 is a flow chart illustrating an example technique to manufacture the luer assembly of FG. 1.

### DETAILED DESCRIPTION

A catheter assembly (e.g., an over-the-wire catheter assembly) may include a luer assembly disposed within a handle of the catheter assembly. The luer may be in fluid communication with an inner lumen of the catheter assembly. A clinician may use the luer of the luer assembly to insert a guidewire into the inner lumen of the catheter assembly. The clinician may also inject a fluid into the inner lumen through the luer assembly, e.g., to flush out any air bubbles present within the inner lumen. In some examples, luers are connected to an inner tube of the catheter (e.g., defining the inner lumen of the catheter assembly) via an adhesive at one or more joints. The inner tube may extend through an outer body or an outer shaft of the catheter assembly and be connected to an array and/or expandable element. External pressures acting on joint(s) between the luer and the inner tube (e.g., during navigation of the catheter assembly within a body of a patient, during flushing of the inner tube) may cause unintended movement of fluids (e.g., blood, air) into or out of the inner tube through the joint(s). In luer assemblies that include the use of adhesives, extensive and multi-stepped inspections may be required, e.g., to reduce a likelihood of leakage across the joint(s) in the luer assembly.

In some examples, this disclosure is directed towards forming a luer assembly with a single monolithic component defining the luer, the outer tube, and the slider. The single monolithic component may be formed by overmolding a molding material (e.g., a polymer resin) over the inner tube. The single monolithic component may be coupled to the inner tube without use of any adhesives.

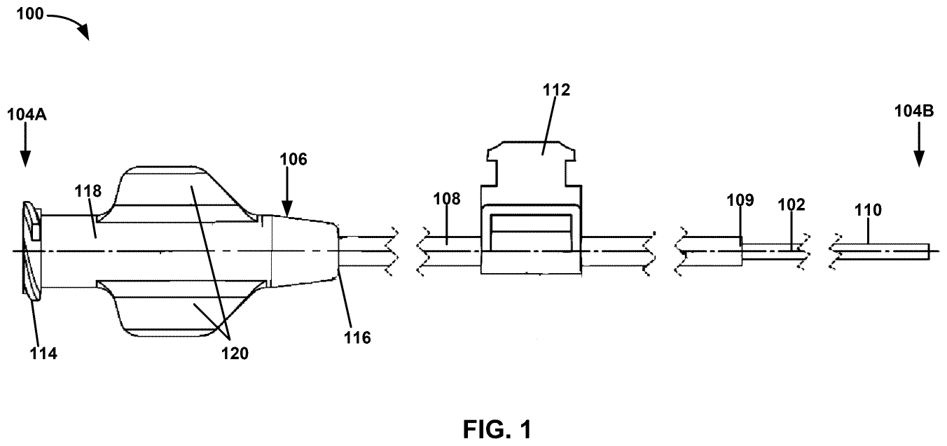

FIG. 1 is a conceptual diagram illustrating an example luer assembly 100. Luer assembly 100 may extend along a longitudinal axis 102 from a proximal end 104A to a distal end 104B. Luer assembly 100 may include luer 106, outer tube 108, sliding mechanism 112 (alternatively referred to herein as “slider 112”) and inner tube 110. Luer 106, outer tube 108, and/or slider 112 may be overmolded over inner tube 110. Luer 106, outer tube 108, and/or slider 112 may be affixed to or disposed over inner tube 110 without use of any adhesives. That is, overmolding may refer to an injection molding process that creates a single solid piece by molding two or more components over each other, which may mean that adhesives are not needed to affix luer 106, outer tube 108, slider 112, and/or inner tube 110 with one another.

Inner tube 110 may extend along longitudinal axis 102 to distal end 104B. Inner tube 110 may define an inner lumen (not pictured in FIG. 1) and may be sized to retain a guidewire. Inner tube 110 may define a uniform or varied diameter along longitudinal axis 102. Inner tube 110 may be formed from: one or more metallic alloys, one or more biocompatible polymers, one or more shape memory materials, or the like.

A distal end of inner tube 110 may be coupled to a therapy delivery element on a distal end of the catheter. The clinician may retract or deploy the therapy delivery element (e.g., an expandable element defining the therapy delivery element) by distally advancing or proximally retracting inner tube 110 along longitudinal axis 102. In one or more examples, because inner tube 110 is overmolded with luer 106, outer tube 108, and slider 112, to distally advance or proximally retract inner tube 110 may mean that all of luer 106, outer tube 108, and slider 112, along with inner tube 110, are distally advanced or proximally retracted. That is, due to the overmolding, luer 106, outer tube 108, slider 112, and inner tube 110 all move together when inner tube 110 is to be distally advanced or proximally retracted.

Luer 106 may be coupled to inner tube 102 at or around proximal end 104A of luer assembly 104A. Luer 106 may define luer body 118 extending along longitudinal axis 102 from proximal end 104A to luer distal end 116. Luer body 118 may define an inner cavity (not pictured in FIG. 1) in fluid connection with the inner lumen of inner tube 110. Luer 106 may define a luer hub 114 at or around proximal end 104A. Luer 106 may define one or more protrusions extending radially outwards from luer body 118.

Luer hub 114 may define threaded features around an outer perimeter of luer hub 114. The threaded feature may include male or female threads. The threaded features on luer hub 114 may engage with corresponding features on an adapter or connector, e.g., to fluidically connect a fluid reservoir to the inner cavity of luer 106 and/or to the inner lumen of inner tube 110.

Luer body 118 may be overmolded onto a proximal portion of inner tube 110. For example, luer body 118 may be overmolded around a proximal end of inner tube 110. Luer body 118 may be bonded to an outer surface of inner tube 110. In some other examples, luer body 118 contacts the outer surface of inner tube 110 without being bonded to the outer surface. Luer body 118 may contact the outer surface of inner tube 110 and inhibit movement of inner tube 110 out of luer body 118, e.g., inhibit movement without any use of adhesives. In some examples, luer body 118 retains a valve (not pictured in FIG. 1) between the inner cavity of luer 106 and the inner lumen of inner tube 102. In such examples, the valve may fluidically connect the inner cavity to the inner lumen and inhibit the unintended flow of fluid between the inner cavity and the inner lumen.

Extension(s) 120 may extend radially away from luer body 118. Luer 106 may include one, two, or three or more extensions 120. The clinician may grip onto and/or rotate luer 106 about longitudinal axis 102 via extension(s) 120. Extension(s) 120 may be equally distributed about an outer perimeter of luer body 118. One or more of extension(s) 120 may be circumferentially aligned with one or more other elements on luer assembly 100. For example, as illustrated in FIG. 1, one of extension(s) 120 may be circumferentially aligned with slider 112 of luer assembly 100.

Outer tube 108 may extend distally along longitudinal axis 102 from luer distal end 116 to outer tube distal end 109. Outer tube 108 may define a uniform or varied outer diameter between luer distal end 116 and outer tube distal end 109. Outer tube 108 may be molded over an outer surface of inner tube 110. Outer tube 108 may be bonded to inner tube 110 or may be in contact with inner tube 110. In some examples, an inner surface of outer tube 108 is bonded to or in contact with an outer surface of inner tube 110. Inner tube 110 may be advanced or retracted along longitudinal axis 102 along with or independent of outer tube 108. Outer tube 108 may be integral to luer 106 at or around luer distal end 116.

Slider 112 may be disposed over outer tube 108. Slider 112 may be placed along outer tube 108 at a location distal to luer distal end 116 and proximal to outer tube distal end 109. In some examples, a distal end of slider 112 is flush with outer tube distal end 109. The location may be closer to luer distal end 116 than outer tube distal end 109. Slider 112 may define a projection extending distally away from longitudinal axis 102. Slider 112 may be integrated within the handle of the catheter (e.g., may be disposed within a slot in the handle of the catheter). The clinician may apply a distal force on slider 112 to advance luer assembly 100 (e.g., inner tube 110 of luer assembly 100) distally along longitudinal axis 102 or may apply a proximal force on slider 112 to retract luer assembly 100 (e.g., inner tube 110) proximally along longitudinal axis 102. The clinician may apply directly on slider 112 or via one or more additional elements in engagement with slider 112. In some examples, in response to the force applied on slider 112, the entirety of luer assembly 100 (i.e., including inner tube 110) may move distally or proximally, thereby causing inner tube 110 to advance distally or to be retracted proximally along longitudinal axis 102. In some examples, slider 112 may not move along longitudinal axis 102 independent of inner tube 110.

Luer 106, outer tube 108, and/or slider 112 may be overmolded directly onto inner tube 110 of luer assembly 100. Luer 106, outer tube 108, and/or slider 112 may be integral to each other or may define a single monolithic component. Luer 106, outer tube 108 and/or slider 112 may be formed from a polymer resin such as, but is not limited to, a polycarbonate, a thermoplastic polyetherimide (e.g., Ultem® available from Curbell Plastics, Inc. of Orchard Park, New York), or a thermoplastic elastomer (e.g., Arnitel® available from Envalior of Dusseldorf, Germany).

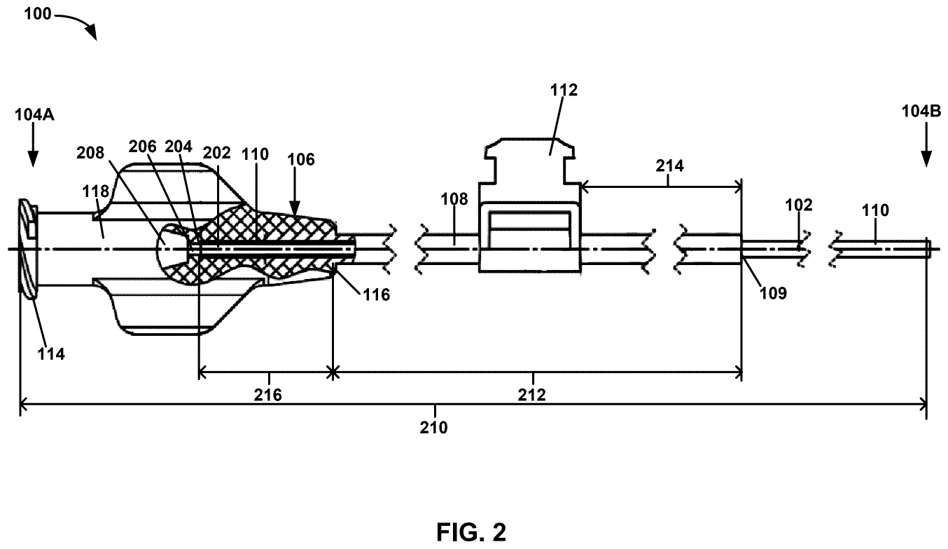

FIG. 2 is a conceptual diagram illustrating an example partially cross-section of luer assembly 100 of FIG. 1. As illustrated in FIG. 2, the cross-section is of luer 106 of luer assembly 100 and is taken along a reference plane extending through longitudinal axis 102.

At least a portion of luer body 118 of luer 106 may be molded over a proximal portion of inner tube 110 (e.g., around proximal end 204 of inner tube 110). Luer body 118 may be bonded to the outer surface of inner tube 110 or may be in contact with the outer surface of inner tube 110. Luer body 118 may contact the outer surface of inner tube 110 across a surface area and may form a seal between an external environment and an inner lumen 202 of inner tube 110.

As illustrated in FIG. 2, luer body 118 may be integral with outer tube 108 (e.g., may form a single monolithic component). For example, at or around luer distal end 116, luer body 118 transitions into outer tube 108, e.g., without any joints between luer body 118 and outer tube 108. Luer body 118 may define a continuous or discrete transition into outer tube 108. Luer body 118 may transition into outer tube 108 at an angle up to 90 degrees. Luer body 118 and outer tube 108 may be formed from a same material and may be formed during a same overmolding process.

Luer body 118 may define an inner cavity 208. Inner cavity 208 may extend distally along longitudinal axis 102 from proximal end 104A towards distal end 104B. Inner cavity 208 may be configured to receive a luer connector, a syringe, a guidewire, or the like. Inner cavity 208 may be formed during the overmolding process. For example, a core pin may be positioned within the mold during the overmolding process to define inner cavity 208.

A distal end of inner cavity 208 may extend to proximal end 204 of inner tube 110, e.g., such that inner cavity 208 is fluidically connected to inner lumen 202. In some examples, a valve 206 may be disposed between inner cavity 208 and inner lumen 202 and may fluidically connect inner cavity 208 to inner lumen 202. Valve 206 may inhibit unintended flow of fluid between inner cavity 208 and inner lumen 202. For example, valve 206 may inhibit the unintended flow of air into inner lumen 202 and/or the unintended flow of blood out of inner lumen 202.

Luer assembly 100 may define various dimensions described below. The dimensions are illustrative of the proportions of luer assembly 100 and are not limiting examples of dimensions of luer assembly 100 as described herein. Any of the dimensions described below may be greater than, equal to, or less than the values attributed below.

Luer assembly 100 may define length 210 along longitudinal axis 102 from proximal end 104A to distal end 104B. Length 210 may be about 135.9 centimeters (cm) (e.g., about 53.5 inches (in)). Outer tube 108 may define a length 212 along longitudinal axis 102 between luer distal end 116 and outer tube distal end 109. An inner surface of outer tube 108 may be in contact with an outer surface of inner tube 110 along at least a portion (e.g., along an entirety) of length 212. Length 212 may be about 17.8 cm (e.g., about 7 in). In some examples, when the clinician inserts at least a portion of luer assembly 100 into a body of a patient, the entire length 212 may be outside the body. In some examples, when the clinician inserts at least a portion of luer assembly 100 into a body of a patient, at least a portion of length 212 is inserted into the body. A distal surface of slider 112 may be separated from outer tube distal end 109 along longitudinal axis 102 by distance 214. Slider 112 may remain outside of the body of the patient as luer assembly 100 is inserted within the body. Distance 214 may be about 4.3 cm (e.g., about 1.7 in).

At or around proximal end 204 of inner tube 110, luer body 118 may be molded over at least a length 216 of inner tube 110. Length 216 may correspond to a portion of inner tube 110 between proximal end 204 and luer distal end 116 along longitudinal axis 102. Luer body 118 may be in contact with inner tube 110 along an entirety of length 216. Length 216 may be about 1.3 cm (e.g., about 0.5 in). A monolithic component defining luer 106 and outer tube 108 may be in contact with and form seal around inner tube 110 along a longitudinal length from proximal end 204 of inner tube 110 to outer tube distal end 109. The longitudinal length may be a sum of lengths 212 and 216. The longitudinal length may increase a contact surface area between luer 106, outer tube 108, and inner tube 110 (e.g., compared to other luer assemblies), e.g., thereby increasing pushability and tensile performance of luer assembly 100. The increased surface area may also improve the seal around inner tube 110, e.g., inhibiting unintended flow of fluid into or out of inner lumen 202 of inner tube 110. As the clinician applies a force on slider 112 (e.g., a distal or proximal force), the force may be transmitted to inner tube 110 across the contact surface area (e.g., to advance or retract luer assembly 100). The increased surface area may reduce pressure on the contact area resulting from the applied force.

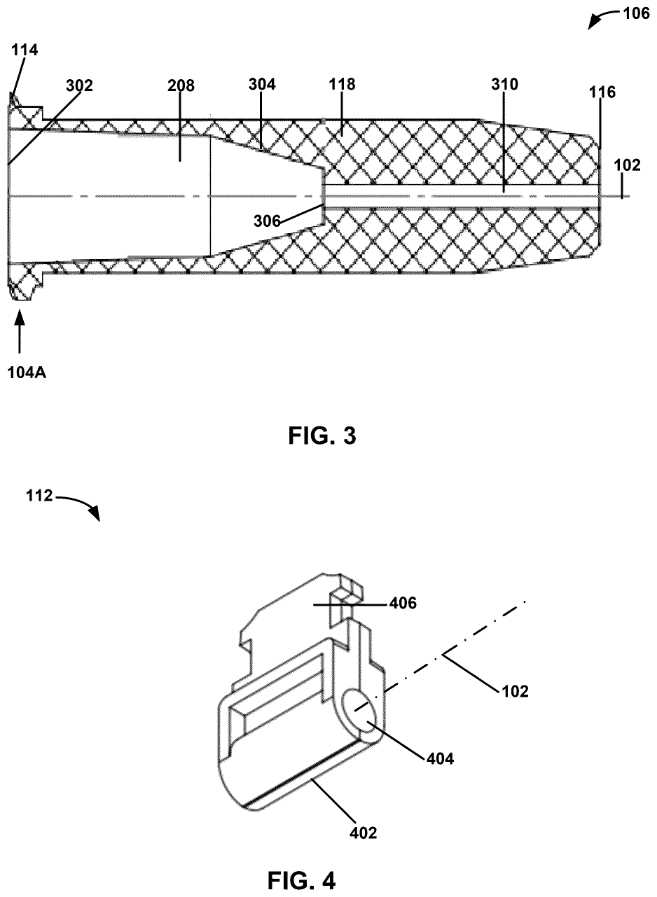

FIG. 3 is a conceptual diagram illustrating an example cross-section of luer 106 of luer assembly 100 of FIG. 1. Luer 106 may include luer body 118 extending distally along longitudinal axis 102 from luer hub 114 to luer distal end 116. Luer body 118 may define inner cavity 208 extending distally from luer hub 114 to distal end 306 and an inner lumen 310 extending from distal end 306 to luer distal end 116. In some examples, valve 206 (not pictured in FIG. 3) is disposed within inner cavity 208 and/or inner lumen 310 at or around distal end 306. While FIG. 3 illustrates luer 106 being separate from inner tube 110 of luer assembly 100, luer 106 may be molded over inner tube 110 to form luer assembly 100 and may be permanently affixed to or around inner tube 110. The cross-section illustrated in FIG. 3 is taken along a reference plane extending along longitudinal axis 102.

Inner cavity 208 may be accessible via opening 302 on luer hub 114. The clinician may insert one or more elements (e.g., a guide wire, a luer connector, a syringe, a fluid source) into inner cavity 208 and/or into inner lumen 202 of inner tube 110 (not pictured in FIG. 3) via opening 302. An inner surface 304 of luer body 118 may define an inner surface of inner cavity 208. Inner surface 304 may taper distally towards longitudinal axis 102. Inner surface 304 may define a continuous, variable, or stepwise taper along longitudinal axis 102. The taper of inner surface 304 may help guide the one or more elements towards and/or into inner tube 110.

A manufacturing assembly may form inner cavity 208 by overmolding luer body 118 around a core pin within the mold. When the overmold material is introduced around the core pin, the core pin may define a recess within luer body 118. When the core pin is removed from luer 106 after the overmolding process, the recess previously occupied by the core pin may define inner cavity 208. The core pin may define a taper along an outer surface of the core pin. The taper along the core pin may form the taper of inner surface 304.

Inner lumen 310 may extend along longitudinal axis 102 from a proximal opening at or around distal end 306 of inner cavity 208 to luer distal end 116. Inner lumen 310 may be fluidically connected to inner cavity 208. The manufacturing assembly may form inner lumen 310 by overmolding luer body 118 around length 216 of inner tube 110. In some examples, a core pin is inserted through inner tube 110 during the overmolding process, e.g., to support inner tube 110 during the overmolding process. After the overmolding process is completed, an inner surface of luer body 118 defining inner lumen 310 may be in contact with and/or bonded to an outer surface of inner tube 110. Inner lumen 310 may define an inner diameter less than an outer diameter of inner tube 110. Inner lumen 310 may define a uniform or variable inner diameter.

FIG. 4 is a conceptual diagram illustrating an example slider 112 of luer assembly 100 of FIG. 1. Slider 112 may include a slider body 402 defining slider lumen 404 and extension 406. While FIG. 4 illustrates slider 112 being separate from outer tube 108 and inner tube 110 of luer assembly 100, luer 106 may be molded over inner tube 110 to form luer assembly 100 and may be permanently affixed to or around inner tube 110. The cross-section illustrated in FIG. 3 is taken along a reference plane extending along longitudinal axis 102.

Slider body 402 may define slider lumen 404 extending along longitudinal axis 102. Slider lumen 404 may be sized to retain inner tube 110. In such examples, an inner surface of slider lumen 404 may contact and/or be bonded to an outer surface of inner tube 110. In some examples, slider lumen 404 may be sized to retain inner tube 110 and outer tube 108. In such examples, an inner surface of slider lumen 404 may be permanently affixed to outer tube 108 and outer tube 108 may be in contact with inner tube 110. Inner lumen 404 may extend through an entirety of slider 112. Inner lumen 404 may define an inner diameter equal to an outer diameter of inner tube 110 or of outer tube 108. Slider body 402 may be integral to (e.g., may be a part of a same monolithic component as) outer tube 108.

Extension 406 may extend away from longitudinal axis 102. Extension 406 may be integral to slider body 402. Extension 406 may be configured to be disposed within a slot in a handle of the catheter. The clinician may apply a distal force on extension 406 to advance luer assembly 100 distally along longitudinal axis 102. The clinician may apply a proximal force on extension 406 to retract luer assembly 100 proximally along longitudinal axis 102.

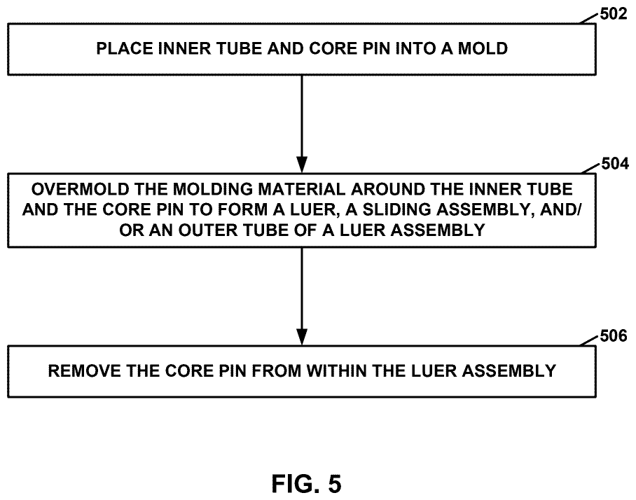

FIG. 5 is a flow chart illustrating an example technique to manufacture luer assembly 100 of FG. 1. The technique illustrated in FIG. 5 may be performed by a manufacturing assembly, a manufacturer, and/or a manufacturing system. While the technique illustrated in FIG. 5 is primarily described herein with reference to luer assembly 100 as illustrated in FIGS. 1-4, the technique may be used to manufacture and/or assemble any other luer assembly described herein.

A manufacturing assembly may place inner tube 110 and a core pin into a mold (502). The mold may define an inner recess configured to retain a volume of molding material during an overmolding process. The molding material may include a polymer resin including, but are not limited to, a polycarbonate, a thermoplastic polyetherimide, or a thermoplastic elastomer. The mold may be shaped to form luer 106, outer tube 108, and slider 112 around inner tube 110. A portion of inner tube 110 (e.g., distal to outer tube distal end 109) may extend out of the mold through an opening in the mold, e.g., to inhibit overmolding of the molding material around the portion of inner tube 110.

The manufacturing assembly may position a core pin within the mold. The manufacturing assembly may align the core pin along a longitudinal axis of inner tube 110 (e.g., longitudinal axis 102) and at a position proximal to inner tube 110. In some examples, the manufacturing assembly may insert a distal end of the core pin into proximal end 204 of inner tube 110, e.g., to block a proximal opening of inner lumen 202 of inner tube 110. Blocking the proximal opening of inner lumen 202 of inner tube 110 may inhibit flow of moldable material into inner lumen 202. The mold may be configured to retain the core pin and inner tube 110 in alignment and/or position, e.g., throughout the overmolding process.

In some examples, such as when luer assembly 100 includes valve 206, the manufacturing assembly places valve 206 along longitudinal axis 102 and between the core pin and proximal end 204 of inner tube 110. Valve 206 may be held in place by core pin and inner tube 110, e.g., without use of adhesive

The manufacturing assembly may overmold the moldable material around inner tube 110 and the core pin to form luer 106, sliding assembly 112, and/or outer tube 108 of luer assembly 100 (504). The manufacturing assembly may insert a moldable material from a reservoir into the inner recess of the mold via one or more gates in the mold. The manufacturing assembly may insert the moldable material into the mold while the moldable material is in a liquid or semi-liquid state. As the moldable material is inserted into the inner recess of the mold, the moldable material may flow around the core pin and inner tube 110 until at least a threshold volume of the inner recess has been filled. Core pin may inhibit the flow of moldable material into inner volume 202 of inner tube 110 as the moldable material is inserted into the mold.

The manufacturing assembly may cool the moldable material (e.g., actively cool, passively cool) within the inner recess of the mold until the moldable material solidifies into a solid component, e.g., forming luer assembly 100. Once the moldable material has solidified, the manufacturing assembly may remove luer assembly 100 from within the mold.

The moldable material within the mold may solidify into a single monolithic component defining luer 106, outer tube 108, and slider 112. In some examples, the single monolithic component defines luer 106 and outer tube 108 and slider 112 is separately affixed to outer tube 108. As the moldable material cools, the moldable material may bond with an outer surface of inner tube 110, e.g., to affix luer body 118 of luer 106 and/or outer tube 108 to inner lube 110 (e.g., without use of an adhesive). In some examples, as the moldable material cools, the moldable material contacts and engages with the outer surface of inner tube 110 (e.g., without forming a bond with the outer surface) to secure inner tube 110 within luer assembly 100.

The manufacturing assembly may remove the core pin from within luer assembly 100 (506). The core pin may occupy and define inner cavity 208 a recess within luer body 118. The manufacturing assembly may remove the core pin from within the inner cavity 208 to allow access into inner cavity 208, e.g., via opening 302. Once core pin is removed, inner cavity 208 may be fluidically connected to inner lumen 202 of inner tube 110 and may allow for the insertion and/or retraction of one or more elements (e.g., a guidewire, a syringe) into or out of inner lumen 202 via inner cavity 208.

Forming, by the manufacturing assembly, luer 106, outer tube 108, and/or slider 112 around inner tube 110 via the overmolding technique may provide several technical advantages over other luer assemblies. Overmolding luer 106, outer tube 108, and/or slider 112 over inner tube 110 may reduce or otherwise eliminate a need to affix the components to inner tube 110 at one or more joints via use of adhesive. The reduction/removal of the need to use of adhesive may simplify the manufacturing process and reduce complexity of luer assembly 100. Overmolding luer 106, outer tube 108, and/or slider 112 over inner tube 110 may increase a contact surface area between inner tube 110 and luer 106, outer tube 108 and/or slider 112 compared to other luer assemblies. The increased contact surface area may improve sealing properties of luer assembly 100, improve pushability of luer assembly 100, improve tensile performance of luer assembly 100, and/or improve sealing of luer assembly 100 (e.g., in response to luer assembly 100 experiencing a positive or a negative pressure). The improved sealing by luer assembly 100 may inhibit unintended flow of fluids into or out of inner tube 110. In some examples, overmolding luer 106, outer tube 108, and/or slider 112 over inner tube 110 may improve a consistency of sealable pressure ranges across a plurality of luer assemblies 110 and/or may improve a reliability of luer assembly 100 under positive or negative pressure conditions.

The following examples are illustrative of the techniques described herein.
Example 1: a luer assembly for a catheter assembly, the luer assembly comprising: an inner tube extending along a longitudinal axis; an outer tube disposed over the inner tube and extending along the longitudinal axis from a proximal end to a distal end; a sliding mechanism coupled to the outer tube; and a luer disposed around the proximal end of the outer tube, wherein the outer tube, the sliding mechanism, and the luer are overmolded over the inner tube.
Example 2: the luer assembly of example 1, wherein the outer tube and the luer are coupled to the inner tube without use of an adhesive.
Example 3: the luer assembly of any of examples 1 and 2, wherein the inner tube defines an outer surface, wherein the outer tube defines an inner surface defining an outer tube lumen, and wherein the inner surface of the outer tube is in contact with the outer surface of the inner tube.
Example 4: the luer assembly of example 3, wherein the inner surface of the outer tube is overmolded directly onto the outer surface of the inner tube.
Example 5: the luer assembly of any of examples 1-4, wherein the outer tube, the sliding mechanism, and the luer define a monolithic component.
Example 6: the luer assembly of any of examples 1-5, wherein the outer tube, the sliding mechanism, and the luer are formed in a single overmolding process.
Example 7: the luer assembly of any of examples 1-6, wherein the inner tube defines an inner lumen, and wherein the luer defines an inner cavity in fluid communication with the inner lumen of the inner tube.
Example 8: the luer assembly of example 7, further comprising a valve disposed within the luer, wherein the valve is disposed between and is in fluid communication with the inner cavity of the luer and the inner lumen of the inner tube.
Example 9: the luer assembly of any of examples 1-8, wherein the outer tube, the sliding mechanism, and the luer are formed from one or more of: a polycarbonate material; a thermoplastic polyetherimide material; or a thermoplastic elastomer.
Example 10: a system comprising: a luer assembly comprising: an inner tube extending from an inner tube proximal end to an inner tube distal end along a longitudinal axis; a luer overmolded around the inner tube proximal end of the inner tube; an outer tube overmolded around the inner tube and extending distally from a luer distal end of the luer; and a sliding mechanism overmolded around the inner tube and coupled to the outer tube, wherein the luer, the outer tube, and the sliding mechanism define a monolithic component.
Example 11: the system of example 10, wherein the monolithic component is coupled to the inner tube without the use of an adhesive.
Example 12: the system of any of examples 10 and 11, wherein the inner tube defines an outer surface, wherein the outer tube defines an inner surface, and wherein the inner surface of the outer tube is overmolded directly onto the outer surface of the inner tube.
Example 13: the system of any of examples 10-12, wherein the luer, the outer tube, and the sliding mechanism are formed in a single overmolding process.
Example 14: the system of any examples 10-13, wherein the inner tube defines an inner lumen, wherein the luer defines an inner cavity, and wherein the inner lumen is in fluid communication with the inner cavity.
Example 15: the system of example 14, wherein the luer assembly further comprises a valve disposed within the luer and in fluid communication with the inner lumen and the inner cavity, and wherein the valve is a separate component from the inner tube and the luer.
Example 16: the system of any of examples 10-15, wherein the monolithic component is formed from one or more of: a polycarbonate material; a thermoplastic polyetherimide material; or a thermoplastic elastomer.
Example 17: a method of manufacturing a luer assembly, the method comprising: placing an inner tube and a center pin within a mold, wherein the inner tube and the center pin are aligned along a longitudinal axis, and wherein a distal end of the center pin is inserted into a proximal end of the inner tube; inserting a polymer resin into the mold and round the inner tube and the center pin to overmold the polymer resin around the inner tube and the core pin to form: a luer around an inner tube proximal end of the inner tube, an outer tube around the inner tube and coupled to a luer distal end of the luer, and a sliding mechanism around the inner tube and coupled to the outer tube; and removing the center pin from within the luer assembly.
Example 18: the method of example 17, wherein the luer defines an inner cavity in fluid communication with an inner lumen of the inner tube, and wherein the center pin defines the inner cavity during the overmolding of the polymer resin around the inner tube.
Example 19: the method of example 18, wherein placing the inner tube and the center pin within the mold comprises: placing a valve between the inner tube and the center pin along the longitudinal axis in a longitudinal arrangement; and placing the inner tube, the center pin, and the valve in the longitudinal arrangement within the mold.
Example 20: the method of any of examples 17-19, wherein the polymer resin comprises one or more of: a polycarbonate material; a thermoplastic polyetherimide material; or a thermoplastic elastomer.

Various examples have been described. These and other examples are within the scope of the following claims.

What is claimed is:

1. A luer assembly for a catheter assembly, the luer assembly comprising:
an inner tube extending along a longitudinal axis;
an outer tube disposed over the inner tube and extending along the longitudinal axis from a proximal end to a distal end;
a sliding mechanism coupled to the outer tube; and
a luer disposed around the proximal end of the outer tube,
wherein the outer tube, the sliding mechanism, and the luer are overmolded over the inner tube.

2. The luer assembly of claim 1, wherein the outer tube and the luer are coupled to the inner tube without use of an adhesive.

3. The luer assembly of claim 1, wherein the inner tube defines an outer surface, wherein the outer tube defines an inner surface defining an outer tube lumen, and wherein the inner surface of the outer tube is in contact with the outer surface of the inner tube.

4. The luer assembly of claim 3, wherein the inner surface of the outer tube is overmolded directly onto the outer surface of the inner tube.

5. The luer assembly of claim 1, wherein the outer tube, the sliding mechanism, and the luer define a monolithic component.

6. The luer assembly of claim 1, wherein the outer tube, the sliding mechanism, and the luer are formed in a single overmolding process.

7. The luer assembly of claim 1, wherein the inner tube defines an inner lumen, and wherein the luer defines an inner cavity in fluid communication with the inner lumen of the inner tube.

8. The luer assembly of claim 7, further comprising a valve disposed within the luer, wherein the valve is disposed between and is in fluid communication with the inner cavity of the luer and the inner lumen of the inner tube.

9. The luer assembly of claim 1, wherein the outer tube, the sliding mechanism, and the luer are formed from one or more of:
a polycarbonate material;
a thermoplastic polyetherimide material; or
a thermoplastic elastomer.

10. A system comprising:
a luer assembly comprising:
an inner tube extending from an inner tube proximal end to an inner tube distal end along a longitudinal axis;
a luer overmolded around the inner tube proximal end of the inner tube;
an outer tube overmolded around the inner tube and extending distally from a luer distal end of the luer; and
a sliding mechanism overmolded around the inner tube and coupled to the outer tube,
wherein the luer, the outer tube, and the sliding mechanism define a monolithic component.

11. The system of claim 10, wherein the monolithic component is coupled to the inner tube without the use of an adhesive.

12. The system of claim 10, wherein the inner tube defines an outer surface, wherein the outer tube defines an inner surface, and wherein the inner surface of the outer tube is overmolded directly onto the outer surface of the inner tube.

13. The system of claim 10, wherein the luer, the outer tube, and the sliding mechanism are formed in a single overmolding process.

14. The system of claim 10, wherein the inner tube defines an inner lumen, wherein the luer defines an inner cavity, and wherein the inner lumen is in fluid communication with the inner cavity.

15. The system of claim 14, wherein the luer assembly further comprises a valve disposed within the luer and in fluid communication with the inner lumen and the inner cavity, and wherein the valve is a separate component from the inner tube and the luer.

16. The system of claim 10, wherein the monolithic component is formed from one or more of:
a polycarbonate material;
a thermoplastic polyetherimide material; or
a thermoplastic elastomer.

17. A method of manufacturing a luer assembly, the method comprising:
placing an inner tube and a center pin within a mold, wherein the inner tube and the center pin are aligned along a longitudinal axis, and wherein a distal end of the center pin is inserted into a proximal end of the inner tube;
inserting a polymer resin into the mold and round the inner tube and the center pin to overmold the polymer resin around the inner tube and the core pin to form:
a luer around an inner tube proximal end of the inner tube,
an outer tube around the inner tube and coupled to a luer distal end of the luer, and
a sliding mechanism around the inner tube and coupled to the outer tube; and
removing the center pin from within the luer assembly.

18. The method of claim 17, wherein the luer defines an inner cavity in fluid communication with an inner lumen of the inner tube, and wherein the center pin defines the inner cavity during the overmolding of the polymer resin around the inner tube.

19. The method of claim 18, wherein placing the inner tube and the center pin within the mold comprises:
placing a valve between the inner tube and the center pin along the longitudinal axis in a longitudinal arrangement; and
placing the inner tube, the center pin, and the valve in the longitudinal arrangement within the mold.

20. The method of claim 17, wherein the polymer resin comprises one or more of:
a polycarbonate material;
a thermoplastic polyetherimide material; or
a thermoplastic elastomer.
